(19) United States
(12) Patent Application Publication
(10) Pub. No.: US 2014/0082757 A1
(43) Pub. Date: Mar. 20, 2014

(54) TRANSFORMED CAENORHABDITIS ELEGANS AND METHOD FOR SCREENING FOR SUBSTANCES REGULATING GLUCOSE METABOLISM USING SAME

(71) Applicants: Seung Jae Lee, Pohang-si (KR); Dong Yeop Lee, Pohang-si (KR)

(72) Inventors: Seung Jae Lee, Pohang-si (KR); Dong Yeop Lee, Pohang-si (KR)

(73) Assignee: POSTECH ACADEMY INDUSTRY FOUNDATION, Pohang-si, Gyeongsangbuk-do (KR)

(21) Appl. No.: 14/119,847

(22) PCT Filed: May 24, 2012
(86) PCT No.: PCT/KR2012/004133

§ 371 Date: Nov. 22, 2013

(30) Foreign Application Priority Data
May 25, 2011 (KR) 10- 2011-0049519

(51) Int. Cl. A01K 67/033; G01N 33/50

(52) U.S. Cl. CPC A01K 67/0336; G01N 33/5085
USPC 800/3; 800/13; 800/21

(57) ABSTRACT

The present invention relates to a method for preparing a transformed Caenorhabditis elegans (C. elegans) which reacts to glucose by exhibiting fluorescence, and to a method for screening for a candidate substance and for a novel gene capable of regulating glucose metabolism and metabolic diseases using the transformed Caenorhabditis elegans. The transformed Caenorhabditis elegans of the present invention is prepared such that changes in glucose may be easily observed on a real-time basis using a fluorescent microscope. The transformed Caenorhabditis elegans of the present invention enables substances for regulating glucose metabolism to be quickly and accurately discovered, and further, may be expected to significantly contribute to studies on a variety of incurable metabolism-related diseases such as obesity, diabetes, and the like.

### TECHNICAL FIELD

The present invention relates to methods of preparing transformed Caenorhabditis elegans (hereinafter abbreviated as C. elegans) responding to sugar to exhibit fluorescence, and screening a candidate substance and a new gene, which can regulate sugar metabolism and a metabolic disease, using the same.

### BACKGROUND ART

C. elegans can be grown in vitro in large numbers at once at a relatively low cost because of its easy gene manipulation and small size. C. elegans is suitable for experimental uses because it takes only approximately 3 days to become an adult through four stages, L1, L2, L3, and L4, after hatching from its egg. C. elegans has a simple structure, with only 959 cells excluding reproductive cells, and is transparent. For these reasons, it is easy to directly observe the inside of C. elegans through a microscope.

In addition, a cell lineage to an adult from an embryo has been completely identified. It is known from the genome project that the genome of C. elegans is three times that of yeast, and ⅓ to ⅕ that of a human. The genome of C. elegans is 40% similar to that of a human, and shares 75% of the 5,000 human disease genes that are known so far. Thus, it is widely considered as a good model system for the study of human diseases.

Recently, as the interest in metabolisms and metabolic diseases has intensively increased, metabolic studies using the genetics of C. elegans are rapidly progressing. Many genes discovered through these studies are also preserved in mammals. For example, homogenous genes for genes important to the metabolism of mammals such as genes for an insulin signaling pathway, genes for an mTOR signaling pathway, and genes for Tubby, SREBP, PPARγ, BAR (bile acid receptor), etc. are present in C. elegans, and their roles in overall metabolism have been actively studied.

Sugar is an essential source of energy for almost all living organisms on Earth including humans. However, foods having great amounts of sugars are closely related to obesity, heart diseases and, particularly, type II diabetes causing a disorder of glucose in the blood, and thus can cause serious medical problems.

In Korea, the mortality rate for diabetes in 2003 was 25 of every one hundred thousand people, which had increased by over 47% over the preceding 10 years, compared to 17 of every one hundred thousand people in 1994. Social costs of diabetes in the United States are enormous, estimated to be approximately 98 billion dollars in 1997. Thus, for the sake of reducing the socioeconomic cost of diabetes, studies on diabetes are very important. However, the exact cause of diabetes is difficult to find since diabetes is determined by a complex interaction of genetic and environmental factors. Thus, to identify a network of genes serving in diabetes and find a substance that can regulate sugar metabolism, the use of a simple animal model such as C. elegans can be effective. However, up to now, there have been no studies for screening a candidate gene and a candidate drug involved in sugar metabolism and diabetes using C. elegans.

### DISCLOSURE

### Technical Problem

The present invention is directed to a method of preparing transformed C. elegans whose real-time change of sugar in vivo can be observed by fluorescence using the characteristics of C. elegans, such as a low cost and high efficiency, and screening a candidate gene and a candidate drug, which can regulate sugar metabolism using the same.

Specifically, the present invention established usefulness of the transformed C. elegans as a sugar reporter in vivo by proving that the transformed C. elegans is changed in fluorescence when genes involved in sugar and lipid metabolisms are manipulated.

### Technical Solution

According to an aspect of the present invention, there is provided transformed C. elegans that responds to sugar to exhibit fluorescence in vivo. The sugar includes glucose.

In one embodiment of the present invention, the transformed C. elegans has far-3::GFP DNA prepared by attaching a green fluorescence protein (GFP) to a gene whose expression is increased by sugar uptake, i.e., far-3, which is integrated on a chromosome thereof.

According to another aspect of the present invention, there is provided a method of preparing transformed C. elegans including integrating far-3::GFP DNA on a chromosome thereof by UV radiation.

According to still another aspect of the present invention, there is provided a method of screening a candidate gene and a candidate drug, which can regulate sugar metabolism, using transformed C. elegans that responds to sugar to exhibit fluorescence in vivo.

In one embodiment of the present invention, the screening method includes suppressing the expression of a gene for regulating sugar metabolism using RNA interference (RNAi) as soon as the transformed C. elegans uptakes sugar.

### Advantageous Effects

Transformed C. elegans is prepared to easily observe the change in glucose in real time by an actual fluorescence microscope, and thus a sugar metabolism regulatory substance can be found rapidly and exactly. In addition, highly effective studies are possible at a low cost due to the characteristics of C. elegans. Thus, the present invention is expected to considerably contribute to studies for treating various incurable diseases relating to metabolism such as obesity, diabetes, etc.

### DESCRIPTION OF DRAWINGS

FIG. 1 is a schematic diagram illustrating DNA integration in the preparation of transformed C. elegans;

FIG. 2A shows that far-3::GFP transformed C. elegans grown in a medium containing 2% glucose exhibits brighter fluorescence than the control, and FIG. 2B shows a quantified result of the fluorescence;

FIG. 3A shows that the fluorescence increased by glucose is further increased than the control when glycolysis is interrupted by gpi-1 RNAi in the presence of glucose, and FIG. 3B shows a quantified result of the fluorescence;

FIG. 4A shows that the fluorescence increased by glucose is further increased than the control when synthesis of a fatty acid is interrupted by pod-2 RNAi or fasn-1RNAi in the presence of glucose, and FIG. 4B shows a quantified result of the fluorescence; and

FIG. 5A shows that the fluorescence increased by glucose is decreased, compared to the control, when the expected expression of a glucose transporter, H17B01.1, is suppressed by RNAi in the presence of glucose, and FIG. 5B shows a quantified result of the fluorescence.

### BEST MODE

The present invention provides transformed C. elegans which responds to sugar to exhibit fluorescence.

The term “C. elegans” used herein is the abbreviation of the scientific name Caenorhabditis elegans, which is a nematode that eats bacteria and lives in soil, and is widely used in genetic, molecular biological, and cell biological experiments.

The term “transformation” used herein means the change in genetic properties of an organism by DNA injected from an external environment.

In addition, the present invention provides a method of preparing transformed C. elegans, which includes integrating far-3::GFP DNA on a chromosome thereof by UV radiation.

The term “green fluorescence protein (GFP)” used herein is a protein expressed in cells to exhibit strong green fluorescence, and widely used in various fields for detecting the expression of a gene, particularly, due to ease of observation.

The term “transformed C. elegans” used herein is a sugar reporter fluorescent C. elegans, prepared by integration of far-3::GFP DNA, which is prepared by attaching a GFP to a gene whose expression is increased by sugar uptake, i.e., far-3.

In addition, the present invention provides a method of screening a candidate gene and a drug for regulating sugar metabolism using transformed C. elegans responding to sugar to exhibit fluorescence in vivo. The screening method uses RNAi.

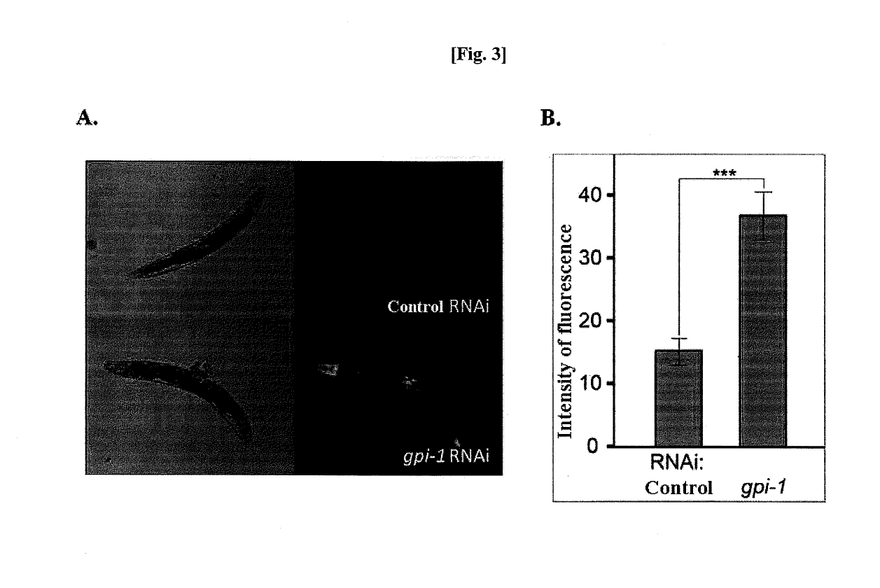

The method includes suppressing the expression of genes of C. elegans using RNAi as soon as the transformed C. elegans uptakes sugar. In one embodiment of the present invention, when lipid and sugar metabolic genes were suppressed, the significant change in intensity of green fluorescence was observed, which shows that a new gene involved in regulation of metabolism can be found using the transformed C. elegans (referred to FIGS. 3 to 5).

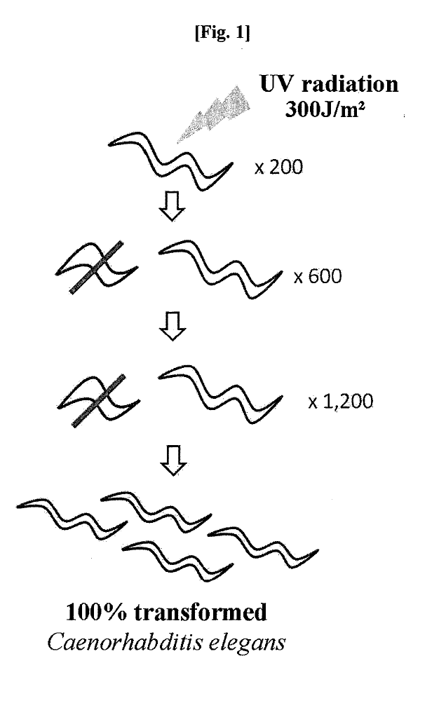

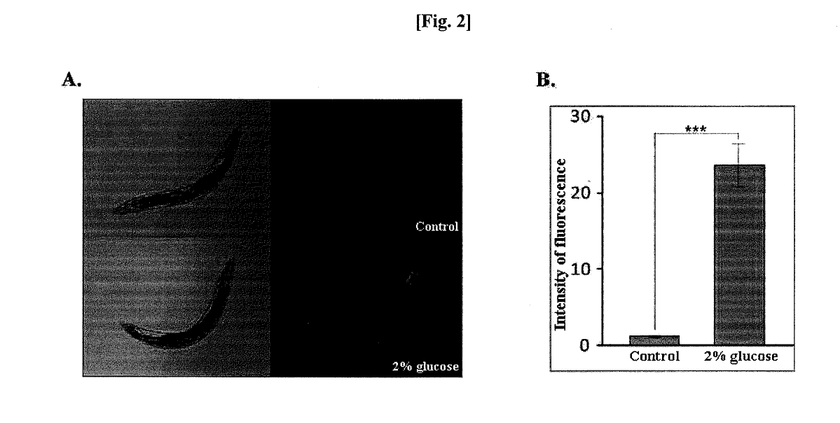

Specifically, the inventors prepared far-3::GFP transformed C. elegans prepared by attaching a GFP to a gene whose expression is increased by sugar uptake, i.e., far-3, to exhibit fluorescence, and then confirmed that, as shown in FIG. 1, far-3::GFP transformed C. elegans exhibited brighter fluorescence than the control when it was grown in a medium containing 2% glucose (refer to FIG. 2).

Subsequently, the inventors confirmed that the fluorescence increased by glucose was further increased than the control when glycolysis was interrupted by gpi-1 RNAi in the presence of glucose (refer to FIG. 3).

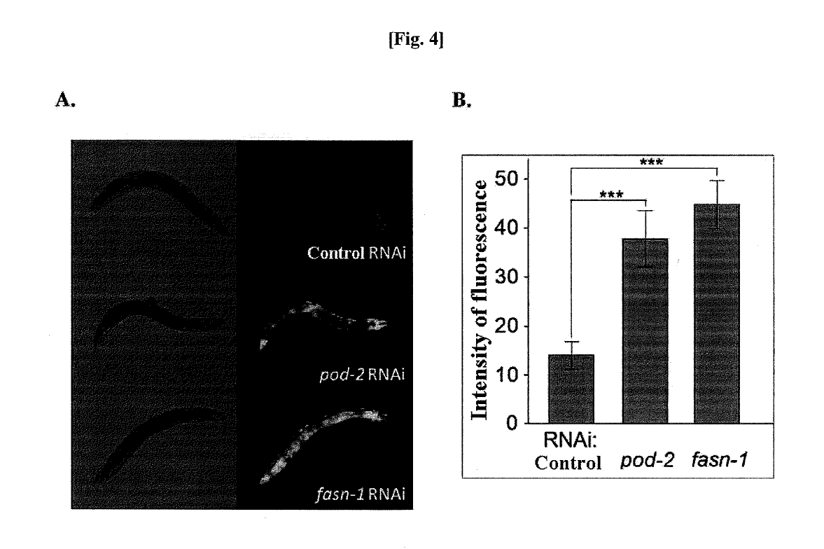

In addition, the inventors confirmed that the fluorescent increased by glucose was further increased than the control when the fatty acid synthesis was interrupted by pod-2 RNAi or fasn-1 RNAi in the presence of glucose (refer to FIG. 4).

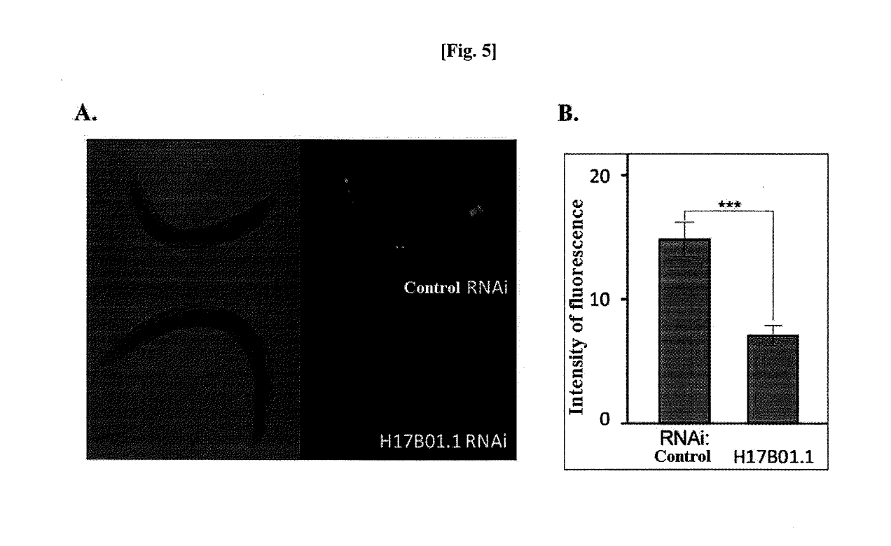

Moreover, the inventors confirmed that the fluorescence increased by glucose was reduced compared to the control when the expected expression of a sugar transporter, H17B01.1, was suppressed by RNAi in the presence of glucose (refer to FIG. 5).

Hereinafter, to help understanding of the present invention, preferable examples will be provided. However, the following examples are merely provided to more easily understand the present invention, not to limit the scope of the present invention.

### EXAMPLES

### Example 1

### Method of Preparing Transformed C. elegans—DNA Integration

Since far-3::GFP DNA inserted into C. elegans is a type that is not included in a chromosome, all of it is not delivered to offspring of C. elegans. For an experiment with such a type of transformed C. elegans, it is difficult to screen transformed C. elegans, and the expression of far-3::GFP is not uniform.

Accordingly, to obtain stable transformed C. elegans, the far-3::GFP DNA input form an external environment was integrated on a chromosome by changing DNA by UV radiation. The DNA integration by UV integration was performed at a very low probability, and thus a rate of success could be increased by UV radiation to a large number of C. elegans.

The far-3::GFP DNA was inserted with a gene recovering a short-body mutant (dpy-5) into a long body, and thus the transformation of C. elegans could be identified with an optical microscope. In the DNA integration, the transformed C. elegans was discriminated in the same manner as described above. As the result of measurement of a frequency of delivering the far-3::GFP DNA to an offspring before the DNA integration, it could be confirmed that the DNA was delivered to approximately 50% of the offspring, and it could be seen that the UV intensity showing approximately 50% viability after UV radiation was 300 J/m².

For a successful experiment for DNA integration, after the UV radiation, over 600 transformed C. elegans were needed, and thus 300 J/m2 of UV radiation was applied to 200 transformed C. elegans in the L4 stage. After 6 days, 600 offspring of C. elegans (F1) born from the UV-treated C. elegans were transferred to 600 plates to produce offspring. After 4 days, two of each plate out of the offspring of C. elegans born from the 600 C. elegans (F2) were transferred into 1,200 plates to produce offspring. After approximately 4 days, the 1,200 plates were examined. Since the transformed C. elegans can be shown 100% if the DNA integration is successfully performed, a plate containing 100% normal-body C. elegans was searched for. 100% normal-body transformed C. elegans was confirmed by identifying the DNA integration by examining fluorescence from a medium containing glucose (referred to FIG. 1).

### Example 2

### Measurement of Fluorescence of Transformed C. elegans of far-3::GFP in the Presence of Glucose

The far-3::GFP transformed C. elegans prepared in Example 1 was grown in a medium containing 2% glucose, and fluorescence was measured by the following method.

Specifically, a drop of 2% agarose was dropped on a slide glass, and covered with another side glass to spread the agarose thinly thereon. The slide glass was removed after the agarose was hardened to form a pad, and a drop of NaN₃ having a concentration of 50 mM was dropped on the agarose pad, and then approximately 15 C. elegans were transferred thereon to be anesthetized. A cover glass was carefully covered without air bubbles, and then fluorescence of far-3::GFP was observed by photographing an image using a fluorescence microscope. As the fluorescence microscope, Zeiss Axio Scope A1 was used. The fluorescence of C. elegans exhibited in the entire body was quantified using an ImageJ program, and the fluorescence of over 20 C. elegans was analyzed by three independent experiments.

As shown in FIG. 2, it was confirmed that the fluorescence was brighter than that of the control, that is, far-3::GFP transformed C. elegans, grown in a medium without glucose (*P<0.05, **P<0.01, ***P<0.001, two-tailed Student's t-test).

### Example 3

### Method of Searching for Sugar Metabolism Gene Using RNAi

The inventors used a Julie Ahringer RNAi bacteria library to suppress the expression of a specific gene and to observe the fluorescence of far-3::GFP. Bacteria obtained from the library were streaked, transferred onto an LB/ampicillin plate, and incubated in an incubator at 37° C. for 12 to 16 hours. One bacteria colony grown on the plate was selected, transferred into a liquid LB/ampicillin medium, and incubated in a shaking culture incubator at 37° C. for 12 hours. After 12 hours, 100 μl of the LB/ampicillin medium in which bacteria were grown was dispensed into an NG medium including 2% glucose and ampicillin, and the bacteria were incubated in an incubator at 37° C. for 12 to 16 hours. For an induction reaction, 50 μl of isopropyl-1-thio-β-D-galactopyranoside (IPTG) having a concentration of 100 mM was dispensed onto the bacteria to react at room temperature for one day, and two adult far-3::GFP transformed C. elegans were transferred onto the plate in which RNAi bacteria were grown to lay eggs. After approximately 12 hours, the adult far-3::GFP transformed C. elegans was removed, and when C. elegans grown from the egg became an adult after three days, the expression of the fluorescence was observed using a method of measuring the fluorescence of the far-3::GFP by a fluorescence microscope.

### Example 3-1

### Use of gpi-l RNAi

The inventors observed the change of intensity of fluorescence in C. elegans transformed when the expression of gpi-1 genes known to be related to glycolysis was interrupted using gpi-l RNAi.

When glucose is input into a cell, it is converted into glucose-6-phosphate by a hexokinase, and then is converted into fructose-6-phosphate by a glucose-6-phosphate isomerase (gpi-l). The gpi-l serves an important role in a high step of glycolysis and thus can interrupt the glycolysis through the suppression of the expression of gpi-l.

As shown in FIG. 3, it was seen that, when the expression of a gpi-1 gene was interrupted, the fluorescence increased by glucose was further increased than in the control, that is, transformed C. elegans, in which the gpi-1 expression was not decreased, in the presence of glucose (*P<0.05, **P<0.01, ***P<0.001, two-tailed Student's t-test).

### Example 3-2

### Use of pod-2 RNAi or fasn-1 RNAi

The inventors observed the change of the intensity of the fluorescence of the transformed C. elegans when the expression of an acetyl-CoA carboxylase (pod-2) or a fatty acid synthase (fasn-1) known to be related to synthesis of fatty acids was interrupted using pod-2 RNAi or fasn-1 RNAi.

The pod-2 is an enzyme located in the highest stage of the synthesis of fatty acids, serving to convert acetyl-CoA into malonyl-CoA. The fasn-1 is involved in the synthesis of fatty acids using acetyl-CoA and malonyl-CoA in the next stage of pod-2. The pod-2 and fasn-1 are major enzymes to regulate the initial stage of the synthesis of fatty acids, which can be considerably suppressed by interrupting pod-2 and fasn-1.

As shown in FIG. 4, it can be seen that the fluorescence increased by glucose was further included when the expression of a pod-2 or fasn-1 gene was interrupted, compared to the control, that is, transformed C. elegans in which the expression of a pod-2 or fasn-1 gene was not increased, in the presence of glucose (*P<0.05, **P<0.01, ***P<0.001, two-tailed Student's t-test).

### Example 3-3

### Use of H17B01.1 RANi

The inventors observed the change of fluorescence intensity in transformed C. elegans when the expected expression of a glucose transporter, H17B01.1, was interrupted using H17B01.1 RANi.

H17B01.1 is expected to be a gene of C. elegans having the most similar base sequence to a human glucose transporter, GLUI1, serving to transport glucose into cells from C. elegans.

As shown in FIG. 5, it was seen that the fluorescence increased by glucose was decreased when the expression of H17B01.1 was interrupted, compared to the control, that is, transformed C. elegans in which the expression of H17B01.1 was interrupted, in the presence of glucose (*P<0.05, **P<0.01, ***P<0.001, two-tailed Student's t-test).

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

### INDUSTRIAL APPLICABILITY

Transformed C. elegans enable a substance for regulating sugar metabolism to be found rapidly and exactly, and furthermore it is expected to considerably contribute to studies for treating various incurable diseases relating to metabolism such as obesity, diabetes, etc.

1. A transformed Caenorhabditis elegans (hereinafter, abbreviated as “C. elegans”), which responds to sugar to exhibit fluorescence in vivo.

2. The transformed C. elegans according to claim 1, further comprising far-3::GFP DNA in which a green fluorescence protein (GFP) is attached to a gene whose expression is increased by sugar uptake, far-3, integrated on a chromosome thereof.

3. A method of preparing transformed C. elegans, comprising:
integrating far-3::GFP DNA on a chromosome thereof by UV radiation.

4. A method of screening a candidate gene and drug for regulating sugar metabolism, which uses transformed C. elegans responding to sugar to exhibit fluorescence in vivo.

5. The method according to claim 4, which comprises:
suppressing the expression of the gene for regulating sugar metabolism using RNA interference (RNAi) as soon as the transformed C. elegans uptakes sugar.
